(19) United States
(12) Patent Application Publication
(10) Pub. No.: US 2017/0196621 A1
(43) Pub. Date: Jul. 13, 2017

(54) SYSTEMS, METHODS, AND DEVICES FOR TISSUE SEALING

(71) Applicant: Versatile Power, Inc., Campbell, CA (US)

(72) Inventors: Noah A. Wilson, Felton, CA (US); David I. Brubaker, San Carlos, CA (US)

(73) Assignee: Versatile Power, Inc., Campbell, CA (US)

(21) Appl. No.: 15/085,764

(22) Filed: Mar. 30, 2016

Related U.S. Application Data
(60) Provisional application No. 62/276,021, filed on Jan. 7, 2016.

(51) Int. Cl. A61B 18/14

(52) U.S. Cl. CPC A61B 18/1445; A61B 2018/00428

(57) ABSTRACT

A system for tissue sealing including first and second electrodes, a drive circuit, a measurement component, a rate component, and a slope regulation component is disclosed. The drive circuit is configured to provide RF energy to the first and second electrodes for application to a load. The measurement component is configured to periodically measure an impedance of the load. The rate component is configured to determine a rate of change for the impedance. The slope regulation component is configured to provide impedance slope regulation. The slope regulation may include adjusting, based on the determined rate of change for the impedance, the RF energy provided by the drive circuit to cause the rate of change for the impedance to follow a predetermined impedance rate.

### RELATED APPLICATIONS

This application claims the benefit under 35 U.S.C. §119(e) of U.S. Provisional Application No. 62/276,021, filed Jan. 7, 2016, entitled “Vessel Sealing Algorithm,” which is incorporated herein by reference in its entirety, including but not limited to those portions that specifically appear hereinafter, the incorporation by reference being made with the following exception: In the event that any portion of the above-referenced application is inconsistent with this application, this application supercedes said above-referenced application.

### TECHNICAL FIELD

The present disclosure relates to tissue sealing and more particularly relates to systems, methods, and devices for improved tissue sealing.

### BRIEF DESCRIPTION OF THE DRAWINGS

FIG. 1 is a schematic diagram illustrating an example system for tissue sealing;

FIG. 2 illustrates a side view of electrodes clamped on a vessel, according to one embodiment;

FIG. 3 is a schematic block diagram illustrating an example sealing control component, according to one embodiment;

FIG. 4 is a schematic flow chart diagram illustrating a method for tissue sealing, according to one embodiment;

FIG. 5 illustrates a graph of current and voltage signals for a vessel sealing process, according to one embodiment; and

FIG. 6 illustrates a graph of impedance during impedance slope regulation, according to one embodiment.

### DETAILED DESCRIPTION

Electro-coagulation, when used for vessel sealing, can seal arteries and create seals capable of maintaining pressures of upwards of 1,000 mmHg (unpublished internal studies). In one embodiment, a seal is made by clamping a vessel between two plate electrodes and applying radio frequency (RF) energy. The applied energy heats the tissue and causes the proteins in the tissue to denature. Denaturing causes the proteins in the tissue to relax hydrogen bonding to themselves, causing a loss in secondary structure that then allows inter-protein binding to occur in the tissue. The amount of binding that occurs during this process determines the strength of the seal.

In some embodiments, pulses of RF energy may be provided to a tissue until the tissue reaches a certain level of electrical impedance. For example, the electrical impedance of the tissue may be periodically measured until a threshold impedance slope or threshold impedance value is exceeded. As used herein, the term impedance is given to mean an electrical impedance, such as resistance in Ohms, of a load. The terms “slope” or “rate of change” for impedance may refer to a rate at which impedance for a load changes. For example, impedance slope or rate of change may be measured or referenced in terms of Ohms per second (Ohms/s). Once the threshold value or slope is reached, the RF energy may be stopped and the sealing procedure may be deemed complete. Some examples of existing approaches are disclosed in the following: U.S. Reissued Pat. No. RE40,388 titled “Electrosurgical Generator With Adaptive Power Control” to David Lee Gines; U.S. Pat. No. 5,540,684 titled “Method And Apparatus For Electrosurgically Treating Tissue” to William L. Hassler, Jr.; U.S. Pat. No. 8,287,528 titled “Vessel Sealing System” to Robert H. Wham et al.; and Bertil Vallfors and Bjorn Bergdahl. Automatically controlled biploar electrocoagulation—“coa-comp”. Neurosurgical Review, 7:187-190, 1984.

However, Applicants have developed significant improvements over existing vessel sealing systems, methods, and algorithms. For example, at least some embodiments presented herein provide for active regulation of the tissue or seal impedance rather than simple triggering of actions based on thresholds. During tissue sealing, the sealing conditions are constantly changing and the active algorithms presented herein may use streams of measurement data to adjust applied power or other aspects of the sealing appropriately. In one embodiment, protocols herein provide flexibility to control the rate of change of the tissue impedance being operated on in real-time. In one embodiment, the impedance slope, or the rate of change of the impedance, may be constant. For example, the impedance may approximate a linear function. In one embodiment, the impedance slope may vary. For example, the impedance over time may approximate a nonlinear function, such as a function that includes oscillating or hyperbolic elements (e.g., sine wave or exponential curve). In one embodiment, a system, method, or device may implement an algorithm that varies the impedance slope dynamically during the seal for reduced adjacent tissue heating (thermal spread) or for improved tissue heating modulation for finer control over tissue charring and sticking.

According to one example embodiment, a system for tissue sealing may include first and second electrodes, a drive circuit, a measurement component, a rate component, and a curve component. The drive circuit is configured to provide RF energy to the first and second electrodes for application to a load. The measurement component is configured to periodically measure an impedance of the load. The rate component is configured to determine a rate of change for the impedance. The curve component is configured to provide impedance slope regulation comprising adjusting, based on the determined rate of change for the impedance, the RF energy provided by the drive circuit to cause the rate of change for the impedance to follow or approximate a predetermined impedance rate.

A detailed description of systems and methods consistent with embodiments of the present disclosure is provided below. While several embodiments are described, it should be understood that this disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments may be practiced without some or all of these details. Moreover, for the purpose of clarity, certain technical material that is known in the related art has not been described in detail in order to avoid unnecessarily obscuring the disclosure.

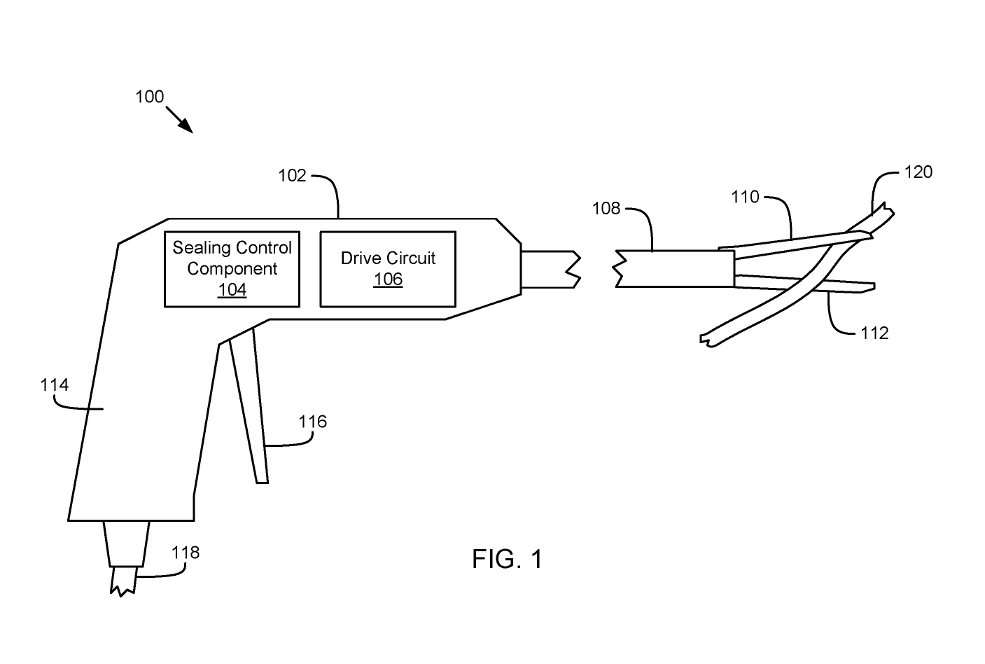

Turning to the figures, FIG. 1 is a schematic diagram illustrating a system 100 for tissue sealing. The system 100 includes a housing 102 that contains internal components including a sealing control component 104 and a drive circuit 106. The housing 102 is connected to a shaft 108. The shaft 108 is connected to the housing 102 at a proximal end and supports a first electrode 110 and a second electrode 112 at a distal end. The system 100 also includes a handle 114 and a lever 116. The system 100 receives power and/or instructions via a cable 118. In one embodiment, an internal power supply, such as a battery, may be included in the housing 102.

The handle 114 may provide a gripping surface or region for a user to manipulate the lever 116, housing 102, shaft 108 and/or electrodes 110, 112. A user may pull on the lever 116 to actuate one or more of the electrodes 110, 112 to clamp onto tissue, such as the vessel 120. Additionally, pulling the lever 116 may initiate the drive circuit 106 and/or the sealing control component 104 to perform a sealing procedure. In one embodiment, the drive circuit 106 is configured to provide electrical energy to the electrodes 110, 112 for application to a load, such as the vessel 120. For example, the drive circuit 106 may provide RF energy to the electrodes 110, 112 via conductors in the shaft 108. The sealing control component 104 may control the drive circuit 106 to provide electrical energy according to a sealing algorithm, such as methods and algorithms discussed herein.

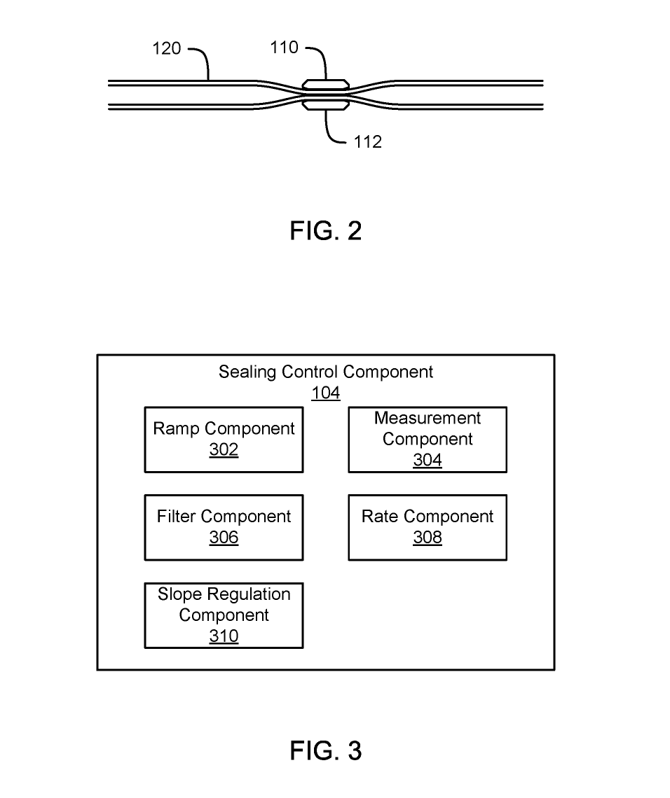

FIG. 2 illustrates a side view of the electrodes 110, 112 that have been closed or clamped on the vessel 120. The electrodes 110, 112 may provide a compressive force on the vessel 120. With the electrodes 110, 112 clamped on the vessel, RF energy may be provided to the electrodes 110, 112, which applies the RF energy across a clamped portion of the vessel 120. The RF energy may heat the vessel 120 to denature proteins in the tissues and allow inter-protein or tissue binding, thus sealing the vessel 120 closed. In one embodiment, the RF energy applied to the electrodes 110, 112, and thus applied to the vessel 120, may be applied in accordance with one or more of the principles disclosed herein.

FIG. 3 is a schematic block diagram illustrating example components of a sealing control component 104. The sealing control component 104 includes a ramp component 302, a measurement component 304, a filter component 306, a rate component 308, and a slope regulation component 310. The components 302-310 are given by way of example only and may not all be included in all embodiments. Each of the components 302-310 may be included in or may be implemented by a sealing control component 104 or part of a separate device, component, or system.

The ramp component 302 is configured to cause a drive circuit to provide an RF energy ramp. For example, the ramp component 302 may control the drive circuit 106 of FIG. 1 to provide an initial ramp of an RF voltage applied to electrodes 110 and 112 and, thus, to a load. In one embodiment, the ramp component 302 causes an RF voltage to ramp, or increase, until a measured current (e.g., measured by the measurement component 304) drops a threshold level below a peak or maximum measured current. For example, the ramping voltage may cause a current through a tissue, which heats the tissue and may increase the impedance of the tissue. As the voltage increases, the impedance may hit such a level that the current begins to drop, even though the voltage is still increasing. At the point the current drops after reaching a maximum current value (e.g., the maximum current value measured during the voltage ramp), the ramp component 302 may end the ramping of the RF energy.

The ramp component 302 may perform the RF ramp at or near a beginning of a tissue or vessel sealing process. In one embodiment, an initial RF energy ramp may start the sealing process and may prepare the tissue or vessel for an impedance slope regulation stage performed by the slope regulation component 310. For example, the initial ramp may reduce an amount of time needed to seal a vessel and/or may improve sealing quality when impedance slope regulation is used.

The measurement component 304 is configured to periodically measure an impedance of a load. For example, the measurement component 302 may periodically obtain impedance measurements of a load (such as a tissue or vessel) clamped between the electrodes 110, 112 of FIGS. 1 and 2. The measurement component 302 may determine the impedance based on an amount of current that results from a currently applied voltage. The measurement component 304 may obtain or perform measurements tens, hundreds, thousands, or more times per second to provide high resolution for how the impedance of a load is changing. In one embodiment, measurements are performed during application of RF energy. The measurements may be performed in real-time so that a current understanding of the actual impedance of the tissue or vessel to be sealed can be obtained.

The filter component 306 is configured to filter impedance measurements obtained by the measurement component 304. For example, the measurements obtained by the measurement component 304 may be subject to noise or errors and may need to be filtered to accurately determine an impedance of a tissue or vessel. In one embodiment, the filter component 306 calculates a windowed mean based on the periodically measured impedance. For example, the filter component 306 may calculate an average of a plurality of measurements and output the average. For example, the filtered data may be output to another component of the sealing control component 104, such as the rate component 308.

The rate component 308 is configured to determine a rate of change for the impedance. For example, the rate component 308 may calculate a real-time slope, or rate of change, of impedance. In one embodiment, the rate component 308 is configured to determine the rate of change for the impedance based on filtered measurements from the filter component 306. For example, the rate of change may indicate how quickly the impedance of a vessel or tissue is changing during sealing of the vessel or tissue.

The rate component 308 may determine whether the impedance is increasing according to a predetermined rate of change. For example, the rate component 308 may compare the measured rate of change with a predetermined rate. In one embodiment, the rate component 308 may store one or more predetermined slopes or rates of change. For example, the rate component 308 may store a table or database of different impedance slopes or impedance rates of change. Based on the type of tissue or vessel being sealed, the rate component 308 may retrieve a different impedance rate, impedance function, or impedance graph. For example, the stored values, functions or graphs may indicate a constant impedance rate and/or a time varying impedance rate. For example, the slope may be a constant slope or a varying slope. In one embodiment, the rate component 308 is configured to compare a current rate of change based on measurements obtained by the measurement component 304 with a predetermined slope, graph, or function from the stored table or database.

The slope regulation component 310 is configured to perform impedance slope regulation. In one embodiment, the slope regulation component 310 is configured to cause an impedance of a tissue or vessel to vary according to (or approximate) a predetermined slope, graph, or function during sealing. In one embodiment, the slope regulation component 310 is configured to vary an amount of RF energy applied to the tissue in order to follow or approximate the predetermined slope or rate of change. For example, the slope regulation component 310 may cause a drive circuit (such as the drive circuit 106 of FIG. 1) to change a voltage, current, and/or frequency applied to the tissue or vessel to be sealed. As a further example, the slope regulation component 310 may increase a voltage, frequency, and/or current in order to increase a rate of change and may reduce a voltage, frequency, and/or current in order to reduce a rate of change for the impedance. In one embodiment, increased energy amounts generally increase rates of change for impedance while reduced energy amounts reduce rates of change.

In one embodiment, the slope regulation component 310 may compare a real-time rate of change (as provided by the rate component 308 based on outputs of the measurement component 304 and/or the filter component 306) with a predetermined rate of change (e.g., from a database, table, or other storage accessed by the rate component 308). If the slope regulation component 310 determines that the real-time impedance slope does not match a predetermined slope, the slope regulation component 310 may determine a change in RF energy to bring the real-time impedance slope in line with the predetermined slope. If the slope regulation component 310 determines that the real-time impedance slope does match a predetermined slope, the slope regulation component 310 may maintain a current RF energy or may calculate a change in RF energy to follow a future slope of the predetermined slope.

In one embodiment, the slope regulation component 310 may include a proportional-integral-derivative (PID) feedback controller. For example, the PID controller may be designed to control RF energy output to approximate or follow an impedance curve for a tissue in real-time. The PID controller may include parameters based on how one or more tissues response to RF energy. In one embodiment, the PID parameters may be stored for each type of tissue or vessel, which may be sealed by the sealing control component 104. The PID controller may be preloaded with the proper parameters to control the driver circuit in order to follow one or more predetermined impedance slopes or curves.

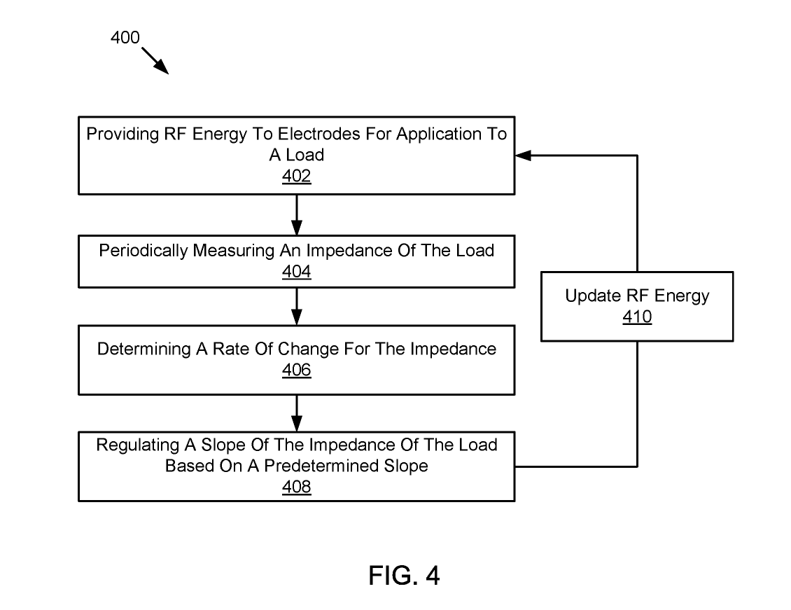

FIG. 4 is a schematic flow chart diagram illustrating an example method 400 for tissue or vessel sealing. The method 400 may be performed by a tissue sealing system or sealing control component, such as the tissue sealing system of FIG. 1 or the sealing control component 104 of FIG. 1 or 3.

The method 400 begins and a drive circuit 106 provides RF energy to the first and second electrodes 110, 112 for application to a load at 402. The RF energy may provide the RF energy to heat the load (e.g., a tissue or vessel) to cause sealing at 402. A measurement component 304 periodically measures an impedance of the load at 404. For example, the measurement component 304 may measure the impedance during application of the RF energy at 404. A rate component 308 determines 406 a rate of change for the impedance. For example, the rate component 308 may calculate a current or instantaneous slope for the impedance in Ohms/s.

Based on the measured slope and a predetermined slope, a slope regulation component 310 regulates the impedance slope at 408. The slope regulation component 310 may regulate by adjusting, based on the determined rate of change for the impedance, the RF energy provided by the drive circuit to cause the rate of change for the impedance to follow a predetermined impedance rate at 408. The predetermine slope may include a constant slope and/or a varying slope.

### Example Implementation

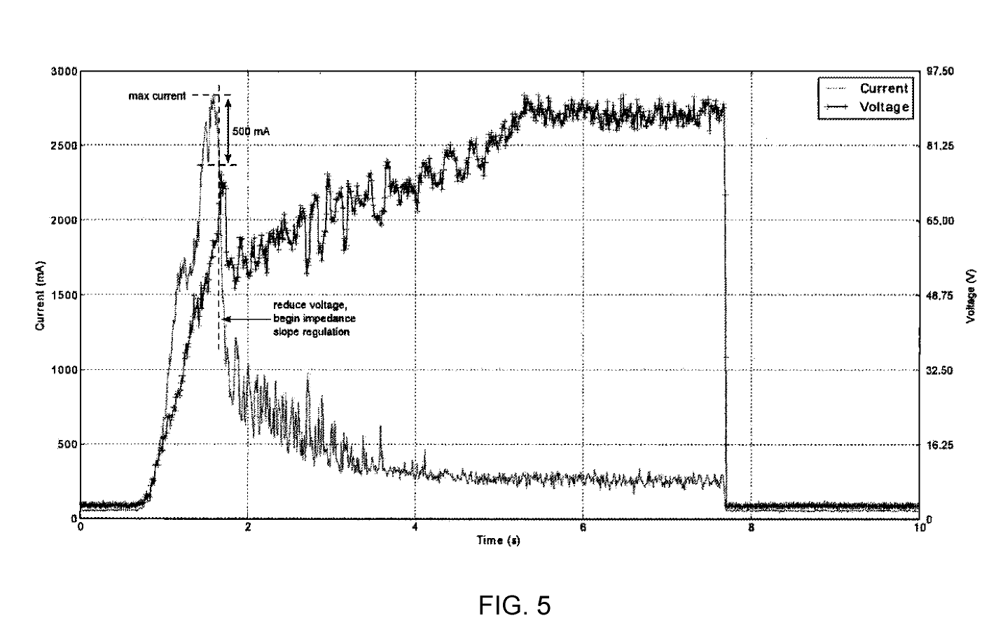

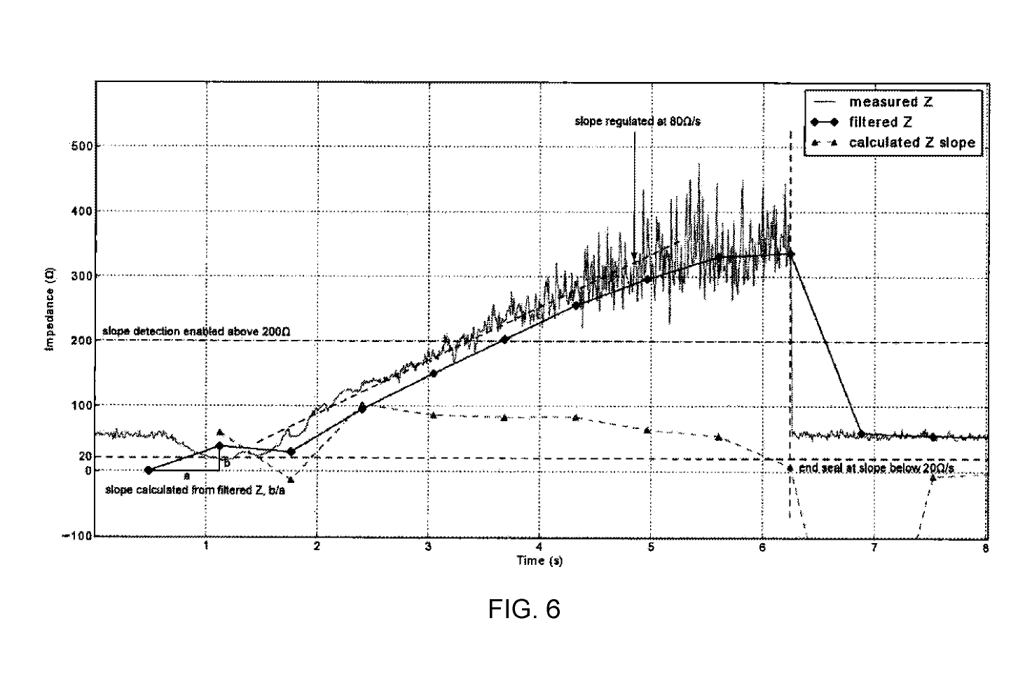

Turning now to FIGS. 5 and 6, one implementation of tissue or vessel sealing will be discussed. During a seal, the system makes voltage (V) and current (I) measurements every Δt milliseconds (ms). From these measurements, the impedance Z of the seal is calculated as:

$${Z}_{n} = \frac{{V}_{n}}{{I}_{n}} , \qquad Equation ( 1 )$$

where Vₙ and Iₙ represent the n-th sample of voltage and current, respectively.

Due to the amount of noise present, the impedance Z is filtered before any conclusions are made concerning the current state of the seal. The system filters the signal by performing a windowed mean on Z every N samples:

$${\overline{Z}}_{m} = {\sum }_{n = {n}_{m}}^{{n}_{m} + N - 1} \frac{{Z}_{n}}{N} , \qquad Equation ( 2 )$$

where N is the number of samples to include in the average and nₘ=[N, 2N, 3N, . . . ]. The filtered signal Z̅ is only updated every N samples and remains constant between updates. For example, N=64 and Δt=10 ms would cause Z̅ₘ to be updated every 640 ms.

The filtered slope of impedance Z̅′̅ can then be calculated from Z̅:

$${\overline{Z}}_{m}^{′} = \frac{{\overline{Z}}_{m} - {\overline{Z}}_{m - 1}}{N \cdot Δ t} , \qquad Equation ( 3 )$$

where Δt represents the time between samples.

At the start of a seal, the system applies RF voltage and ramps its amplitude at a software configurable rate specified in Volts/s. During the voltage ramp, the system monitors the current and maintains a record of the maximum value. After the current has peaked, a subsequent drop in current by some preconfigured amount, which may be specified in milliamps (mA), triggers a change. For example, the change may typically include a reduction in the applied RF voltage. If the current trigger event has not occurred within some configurable time period of applying the voltage ramp, the system may discontinue ramping and reduces the RF voltage, after which it proceeds with the rest of the algorithm (e.g., impedance slope regulation). Current and voltage signals for an illustrative seal are shown in FIG. 5, which may be typical, with the current trigger points labeled. FIG. 5 illustrates a graph of current and voltage signals for a vessel sealing process. In this example, the applied RF voltage ramps until the current drops 500 mA from its maximum recorded value. The system then reduces the applied RF voltage to 50 V and activates impedance slope regulation algorithm. For the remainder of the sealing process, the impedance slope regulator is active, as described below. In one embodiment, throughout the entire sealing process, current and voltage are limited to a predefined maximum. Note that all the values and thresholds used during the vessel sealing process are configurable via software and can be modified to suit the tissue being sealed.

During impedance slope regulation, a PID feedback controller regulates the slope of the impedance to a predefined value, as shown in the example in FIG. 6, by modulating amplitude of the applied RF voltage. In another embodiment, the predefined value may include a predefined function or slope that varies over time. Loss of control over the slope is signaled when the measured slope falls below a defined slope specified in Ohms/s, after which the seal is finished and power is shut off. To ensure a minimum seal quality, the seal is only completed after the impedance has exceeded some minimum impedance. To prevent charring the tissue, the root-mean-square (RMS) RF voltage is limited during impedance slope regulation. More aggressive sealing has been observed at greater impedance slopes. This could lead to different power levels implemented through different impedance slopes. The proportional, integral, and derivative gains of the PID controller can also be set via software. Configuring these gains to suit particular tissue type may improve seal quality and or sealing time.

FIG. 6 is a graph illustrating an example impedance slope for a vessel-sealing algorithm. During impedance regulation, the measured impedance (measured Z), is filtered using a windowed mean (marked with black diamonds). The previous two filtered impedance points are then used to calculated the slope of the impedance (triangle), illustrated by b/a. The system adjusts the amplitude of the applied voltage to maintain a constant slope on the impedance of 80 Ohms/s. After the measured impedance exceeds 200 Ohms, the system begins to watch for the slope to drop below 20 Ohms/s after which the seal is complete and applied voltage is shut off.

To prevent overheating the tissue, the applied RF voltage can be periodically reset to an initial amplitude that was used when the impedance slope regulation began. The interval between RF voltage resets is configurable via software and may typically be set between zero and several seconds, where a zero setting would result in no resets. When enabled, the RF pulsing will periodically reset the RF voltage while the impedance slope regulation algorithm remains active.

### FURTHER EXAMPLES

The following examples pertain to further embodiments.

Example 1 is a system for tissue sealing that includes first and second electrodes, a drive circuit, a measurement component, a rate component, a slope regulation component. The drive circuit is configured to provide RF energy to the first and second electrodes for application to a load. The measurement component is configured to periodically measure an impedance of the load. The rate component is configured to determine a rate of change for the impedance. The slope regulation is component configured to provide impedance slope regulation. The slope regulation may include adjusting, based on the determined rate of change for the impedance, the RF energy provided by the drive circuit to cause the rate of change for the impedance to follow a predetermined impedance rate.

In Example 2, the slope regulation component in Example 1 includes a PID feedback controller.

In Example 3, the system of any of Examples 1-2 further includes a filter component configured to filter measurements provided by the measurement component. The rate component is configured to determine the rate of change for the impedance based on filtered measurements.

In Example 4, the filter component of Example 3 calculates a windowed mean based on the periodically measured impedance.

In Example 5, the drive circuit in any of Examples 1-4 is configured to provide an RF energy ramp, wherein the slope regulation component is configured to adjust the RF energy provided by the drive circuit to cause the rate of change for the impedance to follow the predetermined impedance rate in response to completion of the RF energy ramp.

In Example 6, the RF energy ramp in Example 5 includes increasing an RF voltage until a measured current drops a threshold level below a maximum measured current value.

In Example 7, the predetermined impedance rate in any of Examples 1-6 includes a constant impedance rate.

In Example 8, the predetermined impedance rate in any of Examples 1-7 includes a time varying impedance rate.

Example 9 is a method for controlling application of RF energy to facilitate tissue sealing. The method includes applying RF energy to a region of tissue. The method includes periodically measuring an impedance of the region of tissue. The method includes determining whether the impedance is increasing according to a predetermined rate of change, wherein the predetermined rate of change describes an impedance change during sealing. The method also includes, in response to determining that the impedance is not increasing according to the predetermined rate of change, determining a change in the RF energy to realize the predetermined rate of change. The method includes modifying the application of RF energy based on the determined change, wherein modifying the RF energy comprises increasing or decreasing an energy level for the RF energy.

In Example 10, increasing or decreasing the energy level for the RF energy in Example 9 includes controlling the energy level using a PID feedback controller.

In Example 11, the method of any of Examples 9-10 further includes filtering periodic measurements, wherein determining whether the impedance is increasing comprises determining based on filtered periodic measurements.

In Example 12, filtering in Example 11 includes calculating or determining a windowed mean based on the periodic measurements.

In Example 13, applying the RF energy in any of Examples 9-12 includes providing an RF energy ramp, wherein modifying the application of the RF energy based on the determined change comprises modifying in response to completion of the RF energy ramp.

In Example 14, the RF energy ramp in Example 14 includes increasing an RF voltage until a measured current drops a threshold level below a maximum measured current value.

In Example 15, the predetermined rate of change in any of Examples 9-14 includes a rate of change.

In Example 16, the predetermined impedance rate in any of Examples 9-15 includes a time varying rate of change.

Example 17 is computer readable storage media storing instructions that, when executed by one or more processors, cause the processors to cause a drive circuitry to apply RF energy to a load. The instructions further cause the processors to obtain periodic measurements of an impedance of the load. The instructions further cause the processors to adjust the RF energy applied to the load to cause the impedance to vary over time according to a predetermined rate of change for the impedance. Adjusting the RF energy comprise adjusting an RF voltage, current, or frequency based on the periodic measurements of the impedance of the load.

In Example 18, adjusting the RF energy in Example 17 includes controlling the energy level using a PID feedback controller.

In Example 19, the computer readable storage media in any of Examples 17-18 further store instructions that cause the processors to filter the periodic measurements, wherein adjusting the RF energy applied to the load comprises adjusting based on filtered periodic measurements.

In Example 20, filtering in Example 19 includes calculating or determining a windowed mean based on the periodic measurements.

Example 21 is an apparatus including means to perform a method of any of Examples 9-20.

Various techniques, or certain aspects or portions thereof, may take the form of program code (i.e., instructions) embodied in tangible media, such as floppy diskettes, CD-ROMs, hard drives, a non-transitory computer readable storage medium, or any other machine readable storage medium wherein, when the program code is loaded into and executed by a machine, such as a computer, the machine becomes an apparatus for practicing the various techniques. In the case of program code execution on programmable computers, the computing device may include a processor, a storage medium readable by the processor (including volatile and non-volatile memory and/or storage elements), at least one input device, and at least one output device. The volatile and non-volatile memory and/or storage elements may be a RAM, an EPROM, a flash drive, an optical drive, a magnetic hard drive, or another medium for storing electronic data. One or more programs that may implement or utilize the various techniques described herein may use an application programming interface (API), reusable controls, and the like. Such programs may be implemented in a high-level procedural or an object-oriented programming language to communicate with a computer system. However, the program(s) may be implemented in assembly or machine language, if desired. In any case, the language may be a compiled or interpreted language, and combined with hardware implementations.

It should be understood that many of the functional units described in this specification may be implemented as one or more components, which is a term used to more particularly emphasize their implementation independence. For example, a component may be implemented as a hardware circuit comprising custom very large scale integration (VLSI) circuits or gate arrays, off-the-shelf semiconductors such as logic chips, transistors, or other discrete components. A component may also be implemented in programmable hardware devices such as field programmable gate arrays, programmable array logic, programmable logic devices, or the like.

Components may also be implemented in software for execution by various types of processors. An identified component of executable code may, for instance, comprise one or more physical or logical blocks of computer instructions, which may, for instance, be organized as an object, a procedure, or a function. Nevertheless, the executables of an identified component need not be physically located together, but may comprise disparate instructions stored in different locations that, when joined logically together, comprise the component and achieve the stated purpose for the component.

Indeed, a component of executable code may be a single instruction, or many instructions, and may even be distributed over several different code segments, among different programs, and across several memory devices. Similarly, operational data may be identified and illustrated herein within components, and may be embodied in any suitable form and organized within any suitable type of data structure. The operational data may be collected as a single data set, or may be distributed over different locations including over different storage devices, and may exist, at least partially, merely as electronic signals on a system or network. The components may be passive or active, including agents operable to perform desired functions.

Reference throughout this specification to “an example” means that a particular feature, structure, or characteristic described in connection with the example is included in at least one embodiment of the present disclosure. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present disclosure may be referred to herein along with alternatives for the various components thereof. It is understood that such embodiments, examples, and alternatives are not to be construed as de facto equivalents of one another, but are to be considered as separate and autonomous representations of the present disclosure.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive.

Those having skill in the art will appreciate that many changes may be made to the details of the above-described embodiments without departing from the underlying principles of the disclosure. The scope of the present disclosure should, therefore, be determined only by the following claims.

What is claimed is:

1. A system for tissue sealing, the system comprising:
a first electrode and a second electrode;
a drive circuit configured to provide radio frequency (RF) energy to the first and second electrodes for application to a load;
a measurement component configured to periodically measure a current and or voltage and or impedance of the load;
a rate component configured to determine a rate of change for the impedance; and
a slope regulation component configured to provide impedance slope regulation comprising adjusting, based on the determined rate of change for the impedance, the RF energy provided by the drive circuit to cause the rate of change for the impedance to follow a predetermined impedance rate.

2. The system of claim 1, wherein the slope regulation component comprises a proportional-integral-derivative (PID) feedback controller.

3. The system of claim 1, further comprising a filter component configured to filter measurements provided by the measurement component, wherein the rate component is configured to determine the rate of change for the impedance based on filtered measurements.
The system of claim 3, wherein the filter component calculates a windowed mean based on the periodically measured impedance.

4. The system of claim 1, wherein the drive circuit is configured to provide an RF energy ramp, wherein the slope regulation component is configured to adjust the RF energy provided by the drive circuit to cause the rate of change for the impedance to follow the predetermined impedance rate in response to completion of the RF energy ramp.

5. The system of claim 5, wherein the RF energy ramp comprises increasing an RF voltage until a measured current drops to a threshold level below a maximum measured current value.

6. The system of claim 1, wherein the predetermined impedance rate comprises a constant impedance rate.

7. The system of claim 1, wherein the predetermined impedance rate comprises a time varying impedance rate.

8. A method for controlling application of radio frequency (RF) energy to facilitate tissue sealing, the method comprising:
applying RF energy to a region of tissue;
periodically measuring an current, and or voltage, and or impedance impedance of the region of tissue;
determining whether the impedance is increasing according to a predetermined rate of change, wherein the predetermined rate of change describes an impedance change during sealing;
in response to determining that the impedance is not increasing according to the predetermined rate of change, determining a change in the RF energy to realize the predetermined rate of change;
modifying the application of RF energy based on the determined change, wherein modifying the RF energy comprises increasing or decreasing an energy level for the RF energy.

9. The method of claim 9, wherein increasing or decreasing the energy level for the RF energy comprises controlling the energy level using a proportional-integral-derivative (PID) feedback controller.

10. The method of claim 9, further comprising filtering periodic measurements, wherein determining whether the impedance is increasing comprises determining based on filtered periodic measurements.

11. The method of claim 11, wherein filtering comprises calculating or determining a windowed mean based on the periodic measurements.

12. The method of claim 9, wherein applying the RF energy comprise providing an RF energy ramp, wherein modifying the application of the RF energy based on the determined change comprises modifying in response to completion of the RF energy ramp.

13. The method of claim 13, wherein the RF energy ramp comprises increasing an RF voltage until a measured current drops to a threshold level below a maximum measured current value.

14. The method of claim 9, wherein the predetermined rate of change comprises a rate of change.

15. The method of claim 9, wherein the predetermined impedance rate comprises a time varying rate of change.

16. Computer readable storage media storing instructions that, when executed by one or more processors, cause the processors to:
cause a drive circuitry to apply radio frequency (RF) energy to a load;
obtain periodic measurements of an impedance of the load; and
adjust the RF energy applied to the load to cause the impedance to vary over time according to a predetermined rate of change for the impedance, wherein adjusting the RF energy comprise adjusting an RF voltage, current, or frequency based on the periodic measurements of the impedance of the load.

17. The computer readable storage media of claim 17, wherein adjusting the RF energy comprises controlling the energy level using a proportional-integral-derivative (PID) feedback controller.

18. The computer readable storage media of claim 17, further storing instructions that cause the processors to filter the periodic measurements, wherein adjusting the RF energy applied to the load comprises adjusting based on filtered periodic measurements.

19. The computer readable storage media of claim 19, wherein filtering comprises calculating or determining a windowed mean based on the periodic measurements.
